(19) United States
(12) Patent Application Publication
(10) Pub. No.: US 2002/0026217 A1
(43) Pub. Date: Feb. 28, 2002

(54) Apparatus and method for repair of perigraft flow

(76) Inventors: Steven Baker, Sunnyvale, CA (US); Peter Johansson, Campbell, CA (US); Reid Hayashi, Palo Alto, CA (US)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 09/733,580

(22) Filed: Dec. 6, 2000

Related U.S. Application Data
(63) Continuation-in-part of application No. 09/558,180, filed on Apr. 26, 2000.

(51) Int. Cl. A61B 17/06

(52) U.S. Cl. 606/223; 606/225

(57) ABSTRACT

The present invention is directed to an apparatus and method for sealing a graft in a vessel for preventing blood from flowing around the graft and for reducing the pressure within an aneurysm sac. The system generally includes an apparatus and method for safely and easily causing blood to thrombos between a graft and an aneurysm wall or other arteriovenous site.

### BACKGROUND OF THE INVENTION

The present invention is in the general field of surgical instruments. More specifically, the present invention relates to repairing leaking grafts or leaks around endovascular grafts. These devices are useful for preventing the rupture of an aneurysm that has been fitted with a graft that has perigraft flow.

Grafts are generally tubular-shaped or Y-shaped devices that may function to bridge an aneurysm sac and thus prevent blood from flowing through the sac. Clinical studies have shown that some graft implants suffer from perigraft flow (i.e., leaks around the outside of a graft). Such perigraft flow often does not improve over time. If blood were to leak and flow through the sac, thus pressurizing the sac, then the sac might rupture and thus threaten the life of the patient. Consequently, it is useful to employ a device to stop any leaking.

Sometimes, after the flow has been directed through the inserted graft, the pressure in the aneurysm sac may remain high because of collateral flow from other vessels into and out of the sac. These collateral flows often clot and cease by themselves or with the aid of other vascular techniques. However, this clotting does not always result in a reduction in pressure in the aneurysm sac. Therefore, it would be desirable to reduce this pressure within the aneurysm sac.

Embolic materials are commonly delivered via catheters to repair aneurysms, arteriovenous malformations, and fistulas. Some examples of embolic materials include wire coils with or without Dacron fiber, plastic foam particles, and detachable balloons. Some systems employed in the past for occluding arteriovenous sites included pusher-vasoocclusive coil assemblies and various embolic coils that were used in combination with catheters. Many existing coil delivery systems are designed to deliver only one coil at a time, thus slowing down the treatment process. Further, some systems that use saline to deliver emboli are prone to a relatively high risk of causing hypervolemia from over infusing the patient with saline.

References to embolic material herein shall mean: embolic material, emboli, coagulants, thrombotic agents, gel foam, tissue adhesives, or other materials that are suitable for facilitating the repair of leaking grafts or causing thrombosis or occlusion. Such materials may be radiopaque, echogenic, ferromagnetic, or reflective to facilitate visualization of the embolic material and perigraft flow by imaging with fluoroscopy, CT Scan, ultrasound, MRI, angioscopy or other means.

As used herein, the terms “proximal,” “proximal direction,” and “proximally” when used with respect to the invention are intended to mean moving away from or out of the patient. The terms “distal,” “distal direction,” and “distally” when used with respect to the invention are intended to mean moving toward or into the patient. These definitions will apply with respect to apparatus, such catheters, guide wires, and grafts.

What has been needed and heretofore unavailable is a relatively efficient, safe, and easy-to-use apparatus and method for the repair of leaking grafts and the reduction of pressure within an aneurysm sac. The present invention satisfies this need.

### SUMMARY OF THE INVENTION

Briefly, and in general terms, the present invention provides for repairing blood flow around the outside of a graft that is positioned within a blood vessel.

The present invention is directed to an apparatus and method for sealing a graft in a vessel for preventing blood from flowing around the graft and for reducing the pressure within an aneurysm sac. The system generally includes an apparatus and method for safely and easily causing blood to occlude between a graft and an aneurysm wall or other arteriovenous site.

In one aspect of the invention, a system for preventing blood from flowing around the outside of a graft includes a catheter having a proximal end and a distal end positionable within the graft, an elongate tubular member having a proximal end and a distal end for placement in a lumen of the catheter, and a needle for puncturing a wall of the graft. The needle has a proximal end and a distal end positionable within the elongate tubular member and is deflectable such that the needle is angled into a position for piercing the wall of the graft.

A related method for preventing blood from flowing around the outside of a graft, utilizing a system including a catheter, an elongate tubular member, and a needle, includes the step of accessing a leaking site of a vessel with a distal end of the catheter by feeding the catheter along a guide wire that is positioned within the graft. The balloon catheter is inflated and the needle is extended such that the needle pierces a wall of the graft. Embolic material is injected into a space between the outside of the graft and the vessel via the needle. The catheter is then removed.

In yet another aspect of the invention, a system for preventing blood from flowing around the outside of a graft includes a main catheter having a proximal end and a distal end. The system also includes an injection catheter having a proximal end and a distal end positionable within the main catheter, whereby the injection catheter may be advanced relative to the main catheter for piercing a wall of the graft.

A related method for preventing blood from flowing around the outside of a graft, utilizing a system including a main catheter, an injection catheter, and a guide wire, includes the step of advancing the system to a location within the graft. The guide wire is retracted to allow the main catheter to recover to a set geometry and the injection catheter is advanced relative to the main catheter so that a distal end of the injection catheter penetrates a wall of the graft. The injection catheter includes a needle-like injection tip for facilitating the injection of embolic material. Next, embolic material is injected into a space between the outside of the graft and a vessel. The injection catheter is then retracted. The system may then be removed.

In another aspect of the invention, a system for preventing blood from flowing around the outside of a graft includes a guide catheter having a proximal end and a distal end. The system also includes a delivery catheter having a proximal end and a distal end and positionable within the guide catheter. The system further includes a bendable member for stabilizing the guide catheter so that the delivery catheter may be advanced relative to the guide catheter and toward a wall of the graft.

A related method for preventing blood from flowing around the outside of a graft, utilizing a system including a guide catheter, a delivery catheter, and a bendable member for stabilizing the guide catheter, includes the step of advancing the guide wire to a location within the graft. The guide catheter is then advanced to the location within the graft and stabilized with the bendable member. The delivery catheter is then advanced through the guide catheter until the delivery catheter touches a wall of the graft. A guide wire is advanced, wherein a distal end of the guide wire includes a sharp tip for piercing the wall of the graft, relative to the delivery catheter so that the guide wire pierces the wall of the graft. The delivery catheter is then advanced over the guide wire and through the wall of the graft. The guide wire is then removed. Embolic material is then injected via the delivery catheter into a space between the outside of the graft and a vessel, and the system is removed.

In a still further aspect of the invention, a system for preventing blood from flowing to a portion of a vessel includes a graft, the graft being tubular in shape and including a superior end and at least one inferior end. The system also includes a catheter having a proximal end and a distal end positionable within the graft. The system further includes a delivery catheter, wherein the delivery catheter is positionable between the graft and the vessel and facilitates the injection of embolic material. The catheter fits over a guide wire and may be inflated at its distal end.

A related method for preventing blood from flowing to a portion of a vessel, utilizing a system including a graft, the graft being tubular in shape and having a superior end and an inferior end, a balloon catheter, having a proximal end and a distal end positionable within the graft, and a delivery catheter, having a proximal end and a distal end, includes the steps of accessing a desired site with a system via a first guide wire and inflating the balloon catheter. The method further includes the step of implanting the superior end of the graft. A second guide wire is then inserted between the system and an arteriovenous wall. The inferior end of the graft is then deployed and the delivery catheter is advanced over the second guide wire until the distal end of the delivery catheter is between a wall of the graft and a wall of the vessel. The second guide wire is then removed from the delivery catheter. Embolic material is injected via the delivery catheter into a space between the wall of the graft and the wall of the vessel. Next, the delivery catheter is removed and the catheter is deflated. The balloon catheter is then repositioned inside the inferior end of the graft and inflated to implant the inferior end of the graft. The catheter is then deflated and the catheter and first guide wire are removed.

In a further aspect of the invention, a system forpreventing blood from flowing around the outside of a graft includes a delivery catheter having a proximal end and a distal end positionable within the graft. An elongate tubular member is provided having a proximal end and a distal end for placement in a lumen of the catheter. The distal end of the elongate tubular member is sharpened for puncturing a wall of the graft. The distal end of the elongate tubular member further being pre-formed into an arcuate configuration.

A related method for preventing blood from flowing around the outside of a graft utilizes a system including a delivery catheter, an elongate tubular member having a sharpened distal tip, a balloon catheter, and an elongate tubular jacket. The method comprises the steps of advancing the system to a location within the graft, and retracting the jacket and exposing the sharpened distal tip, wherein the elongate tubular member assumes an arcuate configuration at its distal end. The sharpened distal tip of the elongate tubular member is advanced until the tip pierces the wall of the graft. A desired substance is injected via the elongate tubular member into a space between the outside of the graft and a vessel. The system is then removed.

In another aspect of the invention, there is provided a system for delivering coils to a treatment site of a patient. The system includes a delivery catheter having a proximal end and a distal end positionable within a vessel at a treatment site. The catheter further includes a first lumen. A mandrel is provided having a proximal end and a distal end for placement in the first lumen of the catheter. A plurality of hollow coils are supplied for placement on the mandrel.

In a related method, utilizing a system including a delivery catheter, a mandrel, and a plurality of hollow coils, the distal end of the delivery catheter is advanced to a treatment site. A plurality of hollow coils are strung over the mandrel. The mandrel and coils are inserted into the proximal end of the delivery catheter. The mandrel is then removed from the delivery catheter. A guide wire is inserted into the proximal end of the delivery catheter and advanced such that the coils are forced out of the distal end of the delivery catheter.

In another related method, utilizing a system including a delivery catheter, a mandrel, and a plurality of hollow coils, the distal end of the delivery catheter is advanced to a treatment site. A plurality of hollow coils are strung over the mandrel. The mandrel and coils are inserted into the proximal end of the delivery catheter. A tube is then inserted into the proximal end of the delivery catheter. The tube is then advanced over the mandrel such that the coils are forced out of the distal end of the delivery catheter.

In a further aspect, an apparatus for delivering embolic material to a treatment site of a patient includes a delivery catheter having a proximal end and a distal end positionable within a vessel at the treatment site. The catheter further includes a first lumen and a second lumen. A separating member separates the distal portion of the first lumen from the distal portion of the second lumen. A drive pulley is operatively associated with the delivery catheter. A pinch roller is operatively associated with the delivery catheter and proximate the drive pulley. A conveyor line in the shape of a loop is provided. The conveyor line encircles the separating member. The conveyor line is in apposition with the drive pulley and the pinch roller and disposed therebetween.

In a related method for delivering embolic material to a treatment site of a patient, utilizing a catheter having a proximal end and a distal end, the distal end of the catheter is advanced to the treatment site. Embolic material is introduced into the proximal end of the catheter. Portions of embolic material are advanced out of the distal end of the catheter in rapid succession.

In yet another aspect, a system for delivering embolic material to a treatment site of a patient includes a delivery catheter having a proximal end and a distal end positionable within a vessel at the treatment site, the catheter further including a second lumen having a distal portion and a third lumen having a distal portion. The second lumen and the third lumen are in fluid communication at their distal portions.

In a related method, utilizing a catheter having a proximal end and a distal end, the distal end of the catheter to the treatment site. Embolic material is introduced into the proximal end of the catheter. The embolic material is moved distally through the catheter by pumping a liquid into the catheter, wherein at least some of the liquid moves proximally through a lumen and never reaches the patient. Embolic material is then advanced out of the distal end of the catheter.

In another aspect of the invention, an apparatus for delivering embolic material to a treatment site of a patient includes a catheter having a proximal end and a distal end positionable within a vessel at the treatment site. The catheter further includes a lumen having a distal portion. A screw is disposed within the lumen, the screw operatively associated with the catheter.

In a related method, embolic material is introduced into the lumen of the catheter. The screw is rotated such that at least some of the embolic material is forced out of the distal end of the catheter.

In a still further aspect there is provided a system for delivering embolic material to a treatment site of a patient, including a catheter having a proximal end and a distal end positionable within a vessel at the treatment site. The catheter further includes a lumen having a distal portion. A clip is provided for holding embolic material. The clip in fluid communication with the catheter. An elongate member is provided that may be slidably disposed within the lumen.

Other features and advantages of the present invention will become more apparent from the following detailed description of the invention when taken in conjunction with the accompanying exemplary drawings.

### BRIEF DESCRIPTION OF THE DRAWINGS

FIG. 1 is partial cross-sectional view of one embodiment depicting a catheter of a repair system in assembled relation with the inflatable member of the catheter in an inflated state.

FIG. 2 is an elevational view, with a portion of anatomy shown partially in cross-section, depicting the repair system of FIG. 1 in use.

FIG. 3 is a cross-sectional view taken along line 3-3 of FIG. 1.

FIG. 4 depicts a second embodiment of the repair system.

FIG. 5 depicts the main catheter of the device depicted in FIG. 4.

FIG. 6 depicts the injection catheter of the device depicted in FIG. 4.

FIG. 7 is a cross-sectional view taken along line 7-7 of FIG. 4.

FIG. 8 is an elevational view of the device depicted in FIG. 4 after advancement of the device to a desired location within a graft.

FIG. 9 is an elevational view of the device of FIG. 8 shown delivering embolic material to a space outside of the graft.

FIG. 10 depicts a third embodiment of the repair system.

FIG. 11 is an elevational view, with a portion of anatomy shown partially in cross-section, of the device depicted in FIG. 10 shown piercing a wall of a graft.

FIG. 12 is a cross-sectional view taken along lines 12-12 of the device shown in FIG. 10.

FIG. 13 is an elevational view of the device depicted in FIG. 11 shown delivering embolic material to a space outside of the graft.

FIG. 14 is an elevational view of one component of a fourth embodiment of the repair system depicting a catheter.

FIG. 15 is an elevational view, with a portion of anatomy shown partially in cross-section, of the fourth embodiment of the repair system shown during partial deployment.

FIG. 16 is an elevational view of the repair device shown in FIG. 15 during the delivery of embolic material to a space outside of the graft.

FIG. 17 is an elevational view of a fifth embodiment of the repair system.

FIG. 18 is an elevational view, with a portion of anatomy shown partially in cross-section, depicting the repair system of FIG. 17 in use.

FIG. 19 is an elevational view of a sixth embodiment of the repair system.

FIG. 20 shows the system of FIG. 19 with an optional cover.

FIG. 21 depicts embolic coils being delivered by the system of FIG. 19 with the assistance of a guide wire.

FIG. 22 illustrates embolic coils being delivered by the system of FIG. 19 with the assistance of a tube.

FIG. 23 shows an apparatus of a seventh embodiment for delivering embolic material to a treatment site.

FIG. 24 shows a perspective view in partial cross-section of a system of an eighth embodiment for delivering embolic material to a treatment site.

FIG. 25 depicts a cross-sectional view along lines 25-25 of FIG. 24.

FIG. 26 shows an elevational view, in partial cross-section, of an apparatus of a ninth embodiment for delivering embolic material to a treatment site.

FIG. 27 is an elevational view, in partial cross-section, of a system of a tenth embodiment for delivering embolic material to a treatment site.

FIG. 28 is an elevational view, with a portion of anatomy shown partially in cross-section, depicting a bifurcated unibody graft for use with the present invention.

FIG. 29 is a depiction of the graft of FIG. 28 being used in conjunction with a perigraft flow repair procedure.

FIG. 30 is an elevational view, with a portion of anatomy shown partially in cross-section, depicting a bifurcated modular graft for use with the present invention.

### DETAILED DESCRIPTION OF THE PREFERRED EMBODIMENTS

As shown in the exemplary drawings, the present invention is embodied in an apparatus and method for repair of perigraft flow. Like reference numerals indicate like or corresponding elements among the figures.

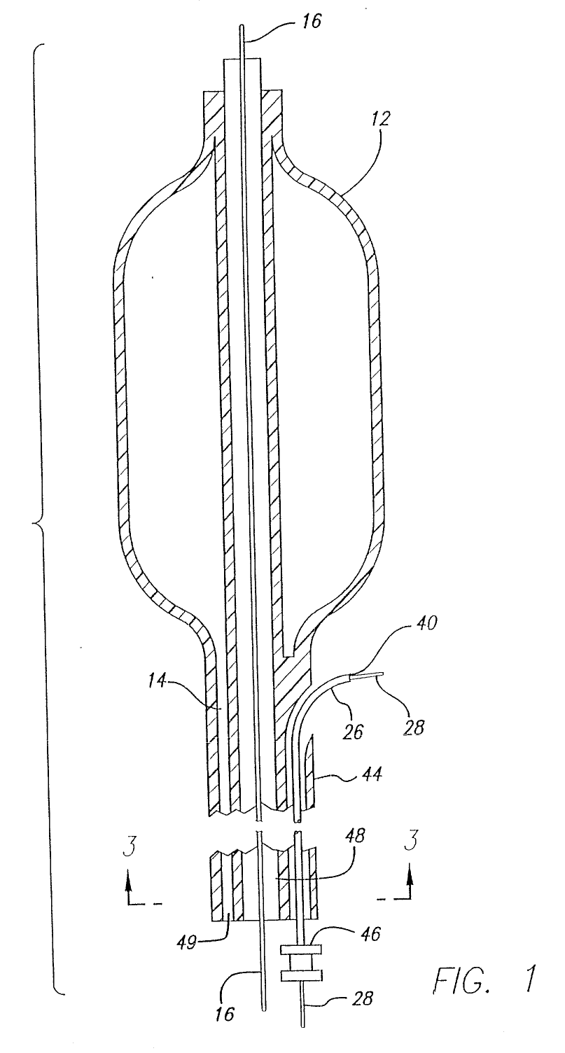

As mentioned previously, some graft implants suffer from perigraft flow that often does not improve over time. If blood were to leak and flow through the sac then the sac might rupture and thus threaten the life of the patient. Therefore, it is useful to employ a device to stop any leaking of blood around the graft. Referring to FIG. 1, which depicts one embodiment of the present invention, means for deflecting, such as catheter 10, or an elongate balloon catheter, is shown in assembled relation in inflated state 12. A balloon inflation lumen 14, used for facilitating inflation of the catheter, is disposed longitudinally throughout the proximal portion of the catheter. A means for advancing, such as guide wire 16, resides inside the catheter and may run throughout the length.

The repair system also includes elongate tubular member 26 that houses means for piercing such as hollow needle 28. Located at the proximal end of the elongate tubular member is luer fitting 46 which is used for facilitating access to the interior of elongate tubular member 26. The elongate tubular member is partially disposed within a lumen of catheter 10. Only the distal end of the elongate tubular member protrudes from within the catheter. The elongate tubular member may be a hypotube and may be constructed from stainless steel or a shape memory alloy. The needle 28 is deflectable and positionable within elongate tubular member 26. It is contemplated that the needle may be preformed into a naturally arcuate configuration but held straight while positioned within the elongate tubular member.

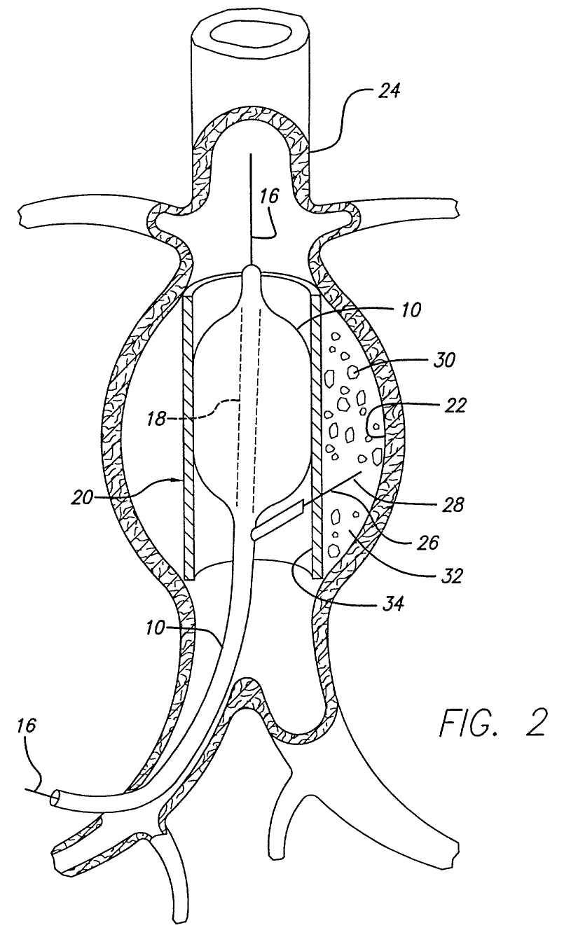

Referring to FIG. 2, needle 28 facilitates the injection of matter such as embolic material 30 into space 32 between wall 34 of graft 20 and wall 22 of vessel 24. The balloon catheter 10 is shown in phantom in deflated state 18 positioned within graft 20. The graft is bridging damaged wall 22 (i.e., an aneurysm sac) of the vessel. The balloon catheter is also shown in inflated state 12. The embolic material can be any of a variety of materials such as coils, foam particles, hydrogels (such as BST-GEL™, available from Biosyntech, 475 Armand-Frappier Boulevard, Montreal, Quebec, Canada H7V 4B3), or polyvinyl alcohols (such as Ivalon™ from Ingenor or Contour™ from Target/Boston Scientific).

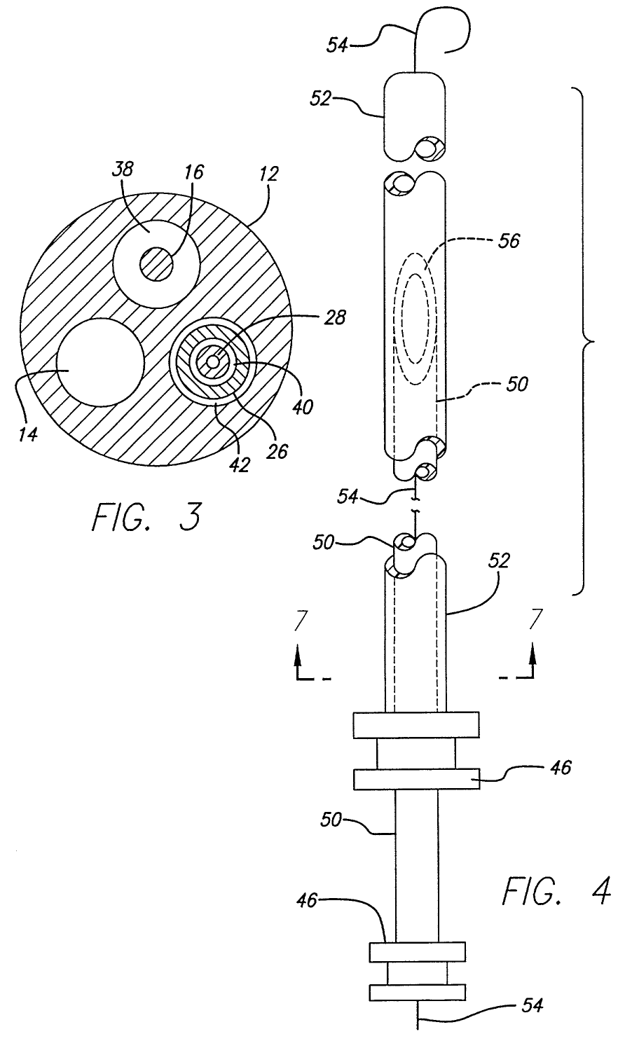

Referring to FIG. 3, as stated, guide wire 16 is positioned within guide wire lumen 38. The guide wire lumen is positioned within inflated catheter 12. The needle 28 is positioned within needle lumen 40 of elongate tubular member 26. The elongate tubular member is, in turn, positioned within elongate tubular member lumen 42 that is housed by support structure 44 (FIG. 1) and runs down the proximal end of catheter 10, where elongate tubular member lumen 42 terminates into an opening. Thus, the guide wire, guide wire lumen, needle 24, needle lumen, elongate tubular member, and elongate tubular member lumen all run parallel to one another within the proximal portion of the catheter.

In use, needle 28 is first inserted into elongate tubular member 26. The elongate tubular member is then fed through elongate tubular member lumen 42 in deflated catheter 18. The guide wire 16 is then advanced through guide wire port 48 (FIG. 1) and using fluoroscopy, to a desired portion of vessel 24 (i.e., through graft 20). Next, the deflated catheter is fed along the guide wire until the needle is in the proper position within the graft. The deflated catheter is then inflated via balloon inflation port 49 and balloon inflation lumen 14. This action angles or deflects the elongate tubular member toward wall 34 of the graft.

At this point, needle 28 is then advanced distally within the elongate tubular member by hand until it exits the distal end of the elongate tubular member. As the needle exits the deflected elongate tubular member 26, the needle punctures the wall of the graft and enters space 32 between the wall of the graft and wall 22 of vessel 24 (e.g., aneurysm sac 22). The embolic material 30 is subsequently injected into the aneurysm sac via fourth means for injecting. A means for injecting may be the needle or another appropriate device. Lastly, inflated catheter 12 is deflated and removed.

The embolic material 30 serves the purpose of preventing blood from flowing between the outside of graft 20 and the aneurysm sac 22. In other words, the leaking graft is repaired so that it leaks no further. Additionally, the embolic material may serve to replace blood that is trapped between the outside of the graft and the aneurysm sac. This is important because sometimes collateral flow into the aneurysm sac from other vessels causes a dangerously high pressure level to exist between the outside of the graft and the aneurysm sac. The injection of the embolic material may serve to lower this pressure, thereby accomplishing the repair of perigraft flow in a relatively safe, easy, and efficient manner.

Preferably, catheter 10 has a proximal diameter ranging from 0.38 cm (0.15 in) to 0.51 cm (0.2 in), a distal diameter ranging from 0.38 cm (0.15 in) to 0.51 cm (0.2 in), and a length ranging from 41 cm (16 in) to 61 cm (24 in). The elongate tubular member 26 may have an inner diameter ranging from 0.356 mm (0.014 in) to 1.778 mm (0.070 in), an outer diameter ranging from 0.457 mm (0.018 in) to 2.286 mm (0.090 in), and a length ranging from 41 cm (16 in) to 61 cm (24 in). The hollow needle 28 may have an inner diameter ranging from 0.254 mm (0.010 in) to 1.016 mm (0.040 in), an outer diameter ranging from 0.305 mm (0.012 in) to 1.27 mm (0.050 in), and a length ranging from 30.5 cm (12 in) to 51 cm (20 in).

Referring to FIG. 4, in a second embodiment of the repair system, injection catheter 50 is positioned within main catheter 52. A guide wire 54 is positioned within the injection catheter. The injection catheter terminates with needle-like injection tip 56 (shown in phantom).

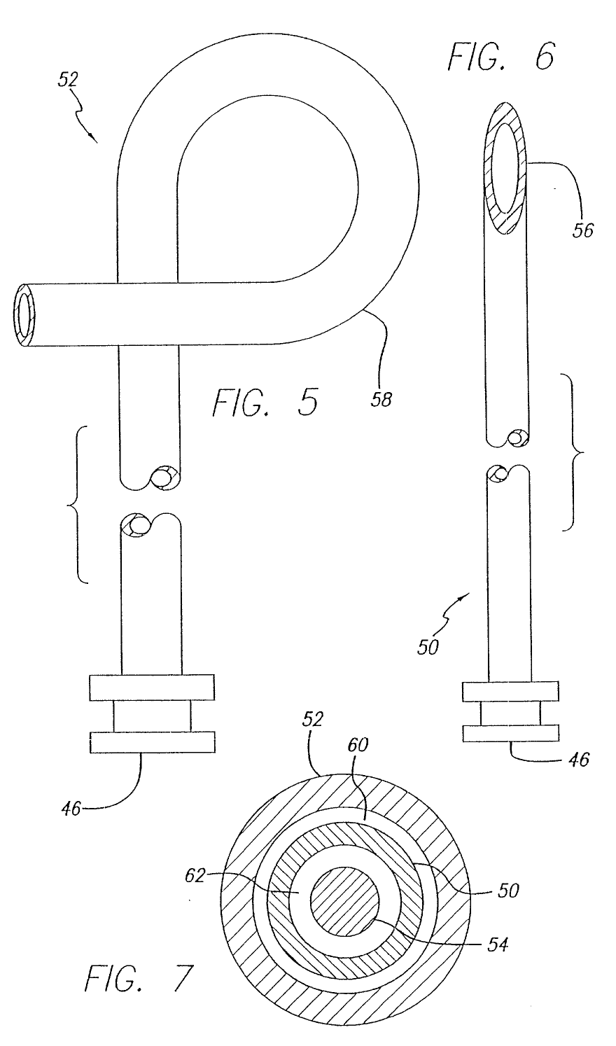

A side view of main catheter 52 is shown in a pigtail configuration in FIG. 5. The main catheter is an elongate tubular member with an inner diameter ranging from 0.051 cm (0.020 in) to 0.18 cm (0.07 in), an outer diameter ranging from 0.08 cm (0.03 in) to 0.23 cm (0.09 in), and a length ranging from 41 cm (16 in) to 61 cm (24 in). The main catheter includes luer fitting 46 at a proximal end for facilitating manipulation of the main catheter and for providing structure for facilitating access to the interior of the main catheter. A distal end 58 of the main catheter is curved (i.e., in a pigtail configuration).

Referring now to FIG. 6, there is shown injection catheter 50. The injection catheter is an elongate tubularmemberwith an inner diameter ranging from 0.038 cm (0.015 in) to 0.102 cm (0.040 in), an outer diameter ranging from 0.051 cm (0.020 in) to 0.152 cm (0.060 in), and a length ranging from 31 cm (12 in) to 51 cm (20 in). The injection catheter similarly includes luer fitting 46 at a proximal end for facilitating manipulation of the injection catheter as well as for providing structure for facilitating access to the interior of the injection catheter. A distal end of the injection catheter is preferably more flexible than main catheter 52 so that the injection catheter may, when inserted within the main catheter, follow a path defined by the main catheter. The distal end of the injection catheter is deflectable, so that it may conform to the pigtail shape of main catheter, and includes needle-like injection tip 56. In one preferred embodiment, the distal end of the injection catheter may be curved.

Turning now to FIG. 7, injection catheter 50 is positioned within main catheter lumen 60 and main catheter 52. The guide wire 54, in turn, is positioned within the injection catheter and injection catheter lumen 62. In one preferred embodiment, the distal end of the guide wire may be curved.

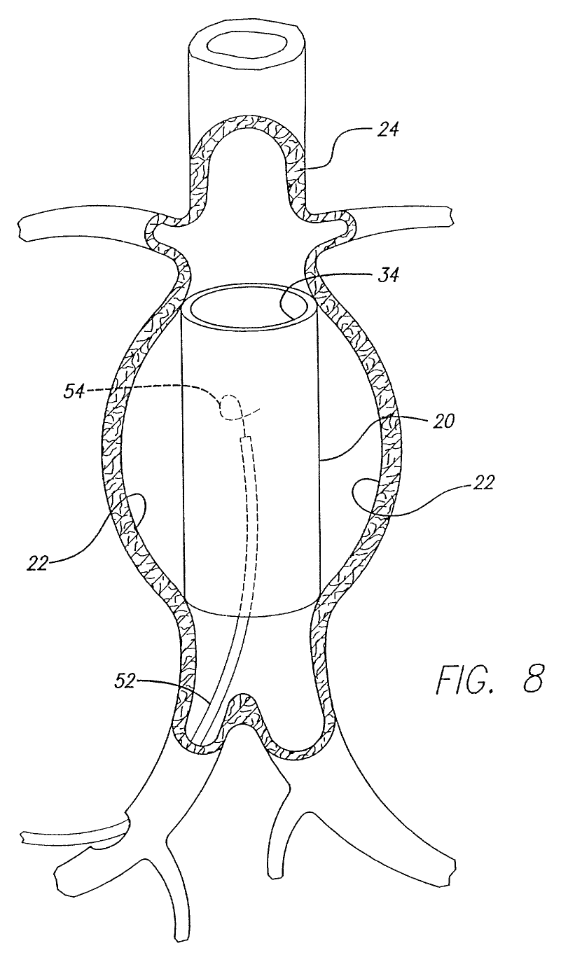

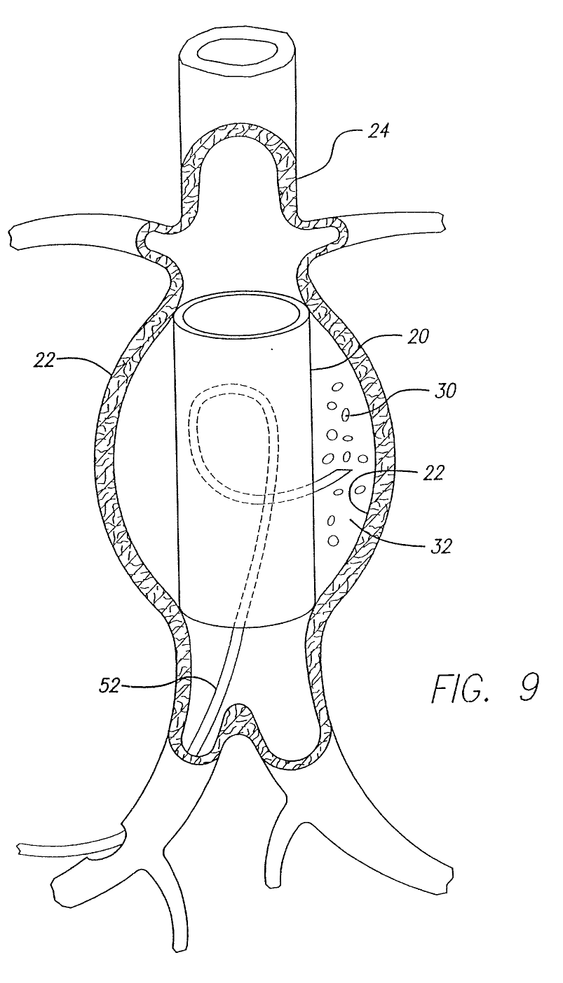

Referring now to FIG. 8, in use, the repair system is advanced to a desired location within graft 20. The guide wire 54 is then retracted to allow main catheter 52 to recover to a set geometry as shown in FIG. 9. The injection catheter 50 is then advanced relative to the main catheter so that needle-like injection tip 56 penetrates wall 34 of the graft. The embolic material 30 is then injected through the injection catheter and into space 22 between the wall of the graft and wall 22 of vessel 24 (e.g., aneurysm sac). The injection catheter is then retracted relative to the main catheter and the system is removed from the patient.

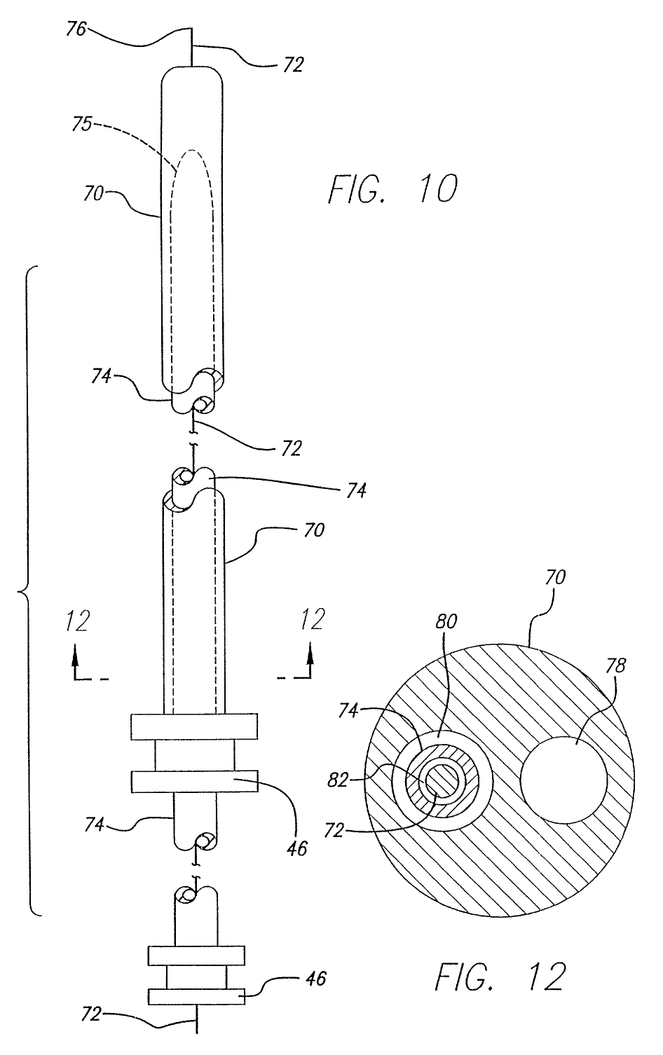

Turning now to a third embodiment of the repair system, there is shown in FIG. 10 a system including guide catheter 70, guide wire 72 (or flexible stylet), and delivery catheter 74. It is contemplated that in one preferred embodiment a hollow needle can be used in place of the delivery catheter. A distal end 75 of the delivery catheter includes a needle-like injection tip for facilitating the injection of embolic material. The delivery catheter is an elongate tubular member with an inner diameter ranging from 0.038 cm (0.015 in) to 0.10 cm (0.04 in), an outer diameter ranging from 0.05 cm (0.02 in) to 0.13 cm (0.05 in), and a length ranging from 31 cm (12 in) to 51 cm (20 in). The delivery catheter is positioned within the guide catheter. The guide catheter is an elongate tubular member with an inner diameter ranging from 0.05 cm (0.02 in) to 0.15 cm (0.06 in), an outer diameter ranging from 0.08 cm (0.03 in) to 0.18 cm (0.07 in), and a length ranging from 41 cm (16 in) to 61 cm (24 in). The guide wire has a sharp tip at distal end 76 for piercing wall 34 of graft 20, and is positioned within the delivery catheter. The guide catheter and the delivery catheter have luer fittings 46 at their proximal ends for facilitating manipulation of the catheters and for providing structure for facilitating access to the interior of the catheters.

Turning now to FIG. 11, the device is depicted, in use, after guide wire 72 and delivery catheter 74 pierce wall 34 of graft 20. The repair system of this embodiment further includes bendable member 77 for stabilizing guide catheter 70. The word “bendable” is used herein to mean able to be bent or expanded. In one preferred embodiment, the bendable member can be a deflecting element for deflecting the guide catheter. The bendable member or deflecting element may take the form of an eccentric balloon 77 (shown schematically in FIG. 11). The eccentric balloon may take the form of a conventional balloon catheter, or the form of any other suitable design. The eccentric balloon may be positioned adjacent guide catheter 70 such that inflation of the eccentric balloon moves the guide catheter so as to deflect the distal end of the guide catheter. In another embodiment, the eccentric balloon may be attached to the guide catheter. Alternatively, the deflection element could include an inner tension device (not shown). The inner tension device could be positioned within the guide catheter or the delivery catheter and when activated causes the catheters to be deflected at their distal ends.

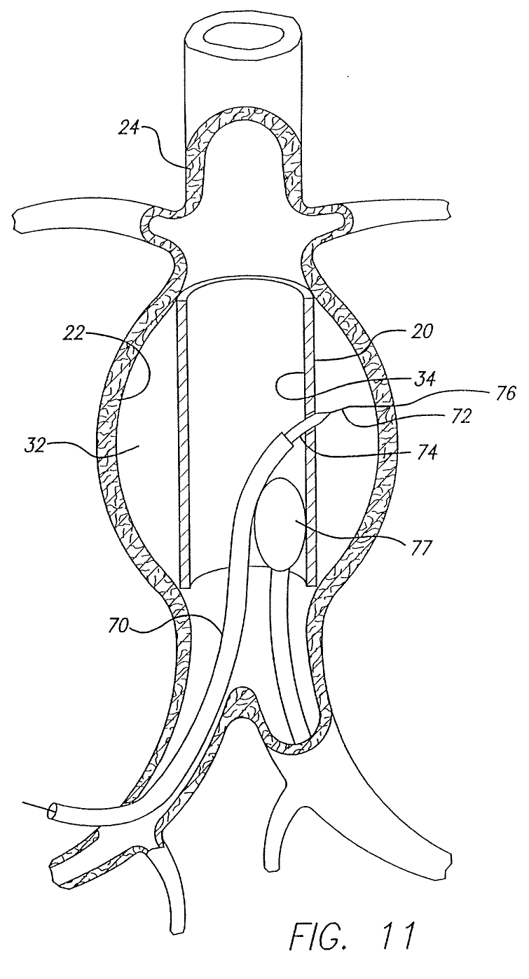

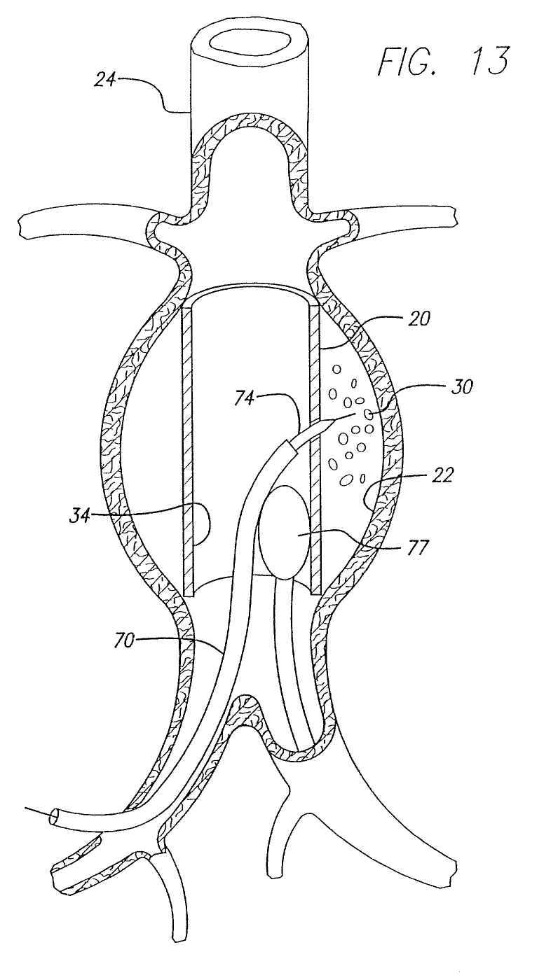

Referring to FIG. 12, there is shown balloon inflation lumen 78 for facilitating the inflation of eccentric balloon 77. The balloon inflation lumen may run within guide catheter 70 as shown. Alternatively, the balloon inflation lumen may be a part of a separate catheter. The eccentric balloon may not be needed to deflect the guide catheter if an inner tension element is implemented instead. The main catheter lumen 80 and delivery catheter lumen 82 are shown running parallel to the balloon inflation lumen. The delivery catheter lumen is positioned within the main catheter lumen.

In use, the guide wire 72 is first advanced to a desired location within graft 20. The guide catheter 70 is then advanced to the desired location. The delivery catheter 74 is then advanced through the guide catheter until it touches wall 34 of the graft. Next, guide 25 wire 72 is advanced relative to the delivery catheter until distal end 76 pierces wall 22 of the graft. The piercing of the graft does not cause a leak. This is because the guide wire penetrates between fibers of the graft and does not cut the fibers. Additionally, thrombosis will help to prevent any leaking.

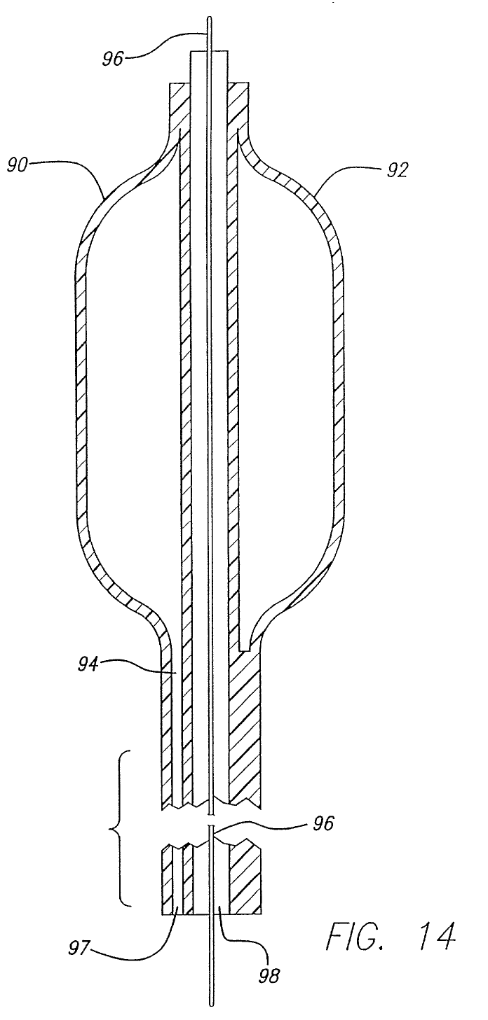

The delivery catheter 74 is then advanced over the guide wire and through the wall of the graft while stabilizing the guide catheter with bendable member 77. The guide wire and guide catheter are then removed. The delivery catheter is then stabilized with the bendable member. The guide wire is removed and embolic material is injected via the delivery catheter into space 32 (i.e., aneurysm sac 22) between the wall of the graft and the wall of vessel 24 (FIG. 13). In one preferred embodiment, one or more of the guidewire (or stylet), delivery catheter, and guide catheter maybe pre-formed into an arcuate configuration. For example, the delivery catheter 74 may be pre-formed into a naturally arcuate configuration, but held straight while in the guide catheter. When the delivery catheter exits from the guide catheter, the delivery catheter assumes its naturally arcuate configuration so that it is in a position to pierce the graft wall. In another preferred embodiment, element 77 can act as a deflection element to deflect the delivery catheter (either directly or by deflecting the guide catheter) into position to pierce the graft wall.

Referring to FIG. 13, embolic material 30 has been injected via delivery catheter 74 into the space between wall 34 of graft 20 and wall 22 (e.g., an aneurysm sac) of vessel 24. Thus, the repair of perigraft flow is accomplished in a relatively safe, easy, and efficient manner.

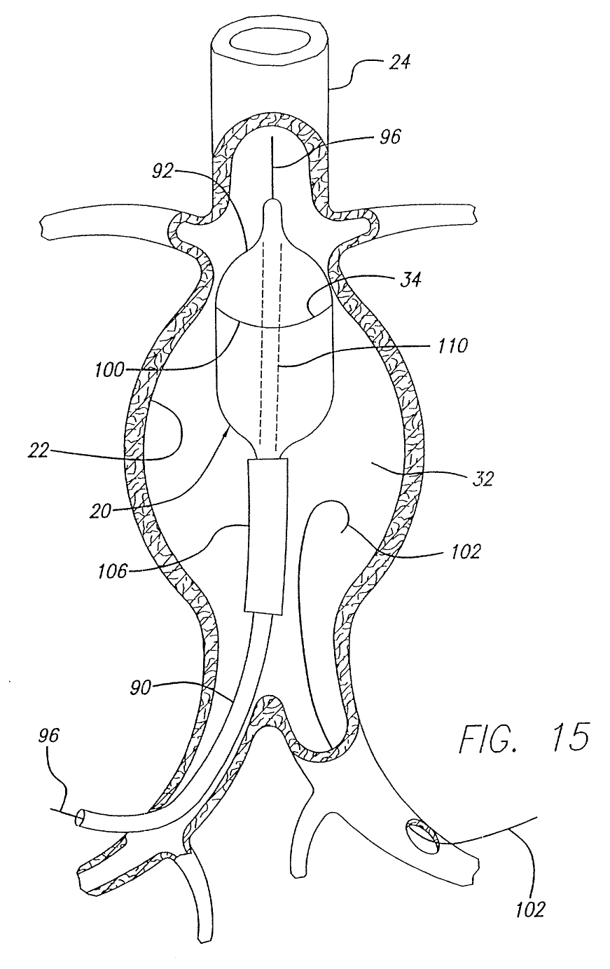

Referring to FIG. 14, in a fourth embodiment there is shown a repair device including catheter 90 in inflated state 92. A balloon inflation lumen 94 and first guide wire 96 run parallel to one another within the catheter. Balloon inflation port 97 is at the proximal end of the balloon inflation lumen. A guide wire port 98 is located at the proximal end of the catheter.

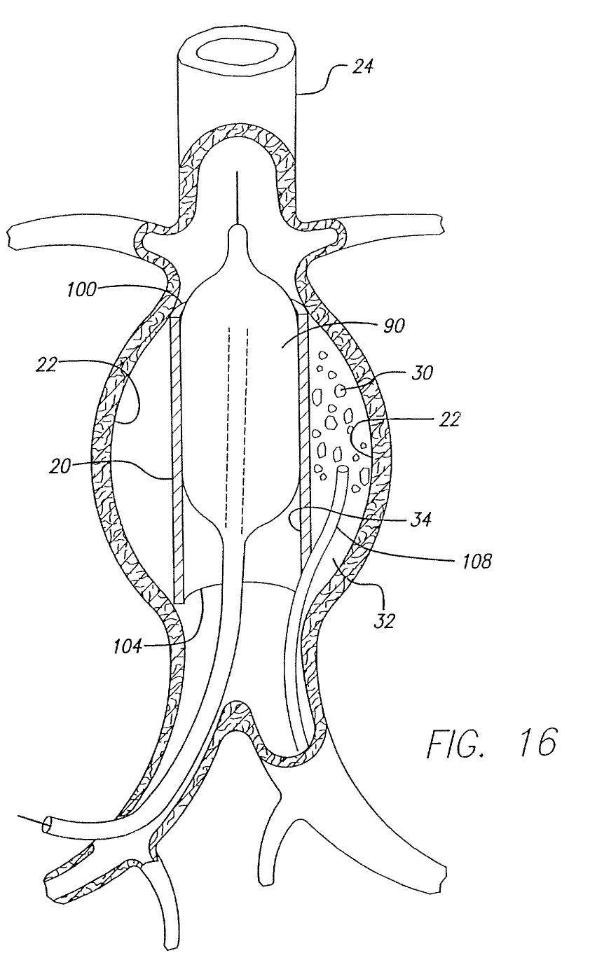

Referring to FIGS. 15 and 16, the repair system further includes second guide wire 102 and elongate delivery catheter 108. The elongate delivery catheter is an elongate tubular member with an inner diameter ranging from 0.13 cm (0.05 in) to 0.30 cm (0.12 in), an outer diameter ranging from 0.15 cm (0.06 in) to 0.38 cm (0.15 in), and a length ranging from 41 cm (16 in) to 51 cm (20 in). The second guide wire and elongate delivery catheter are used in conjunction with the catheter to repair perigraft flow.

In order to repair perigraft flow using this repair system, a desired site is first accessed with the system via first guide wire 96. The catheter 90 is then inflated via balloon inflation port 97 and balloon inflation lumen 94. A superior end 100 of graft 20 is then implanted using conventional means. The second guide wire 102 is thereafter inserted into space 32 between wall 34 of the graft and wall 22 of vessel 24. An inferior end 104 of the graft is subsequently deployed using conventional means, for example, by withdrawing capsule 106 from engagement with the graft. A delivery catheter 108 is subsequently advanced over the second guide wire until the distal end of the delivery catheter is in the space between the wall of the graft and the wall of the vessel. The second guide wire is then removed from the delivery catheter.

As shown in FIG. 16, embolic material 30 is next injected into space 32 between wall 34 of graft 20 and wall 22 (i.e., aneurysm sac) of vessel 24 via delivery catheter 108. A luer fitting, located at the proximal end of the delivery catheter, may be used for facilitating manipulation of the delivery catheter and for providing structure for facilitating access to the interior of the delivery catheter.

After injection, delivery catheter 108 is removed and inflated catheter 92 is deflated and repositioned inside inferior end 104 of the graft. The catheter 90, shown in phantom in deflated state 110, is then inflated to freely implant the inferior end of the graft. Lastly, the inflated catheter is deflated and removed. Thus, the repair of perigraft flow is accomplished in a relatively safe, easy, and efficient manner.

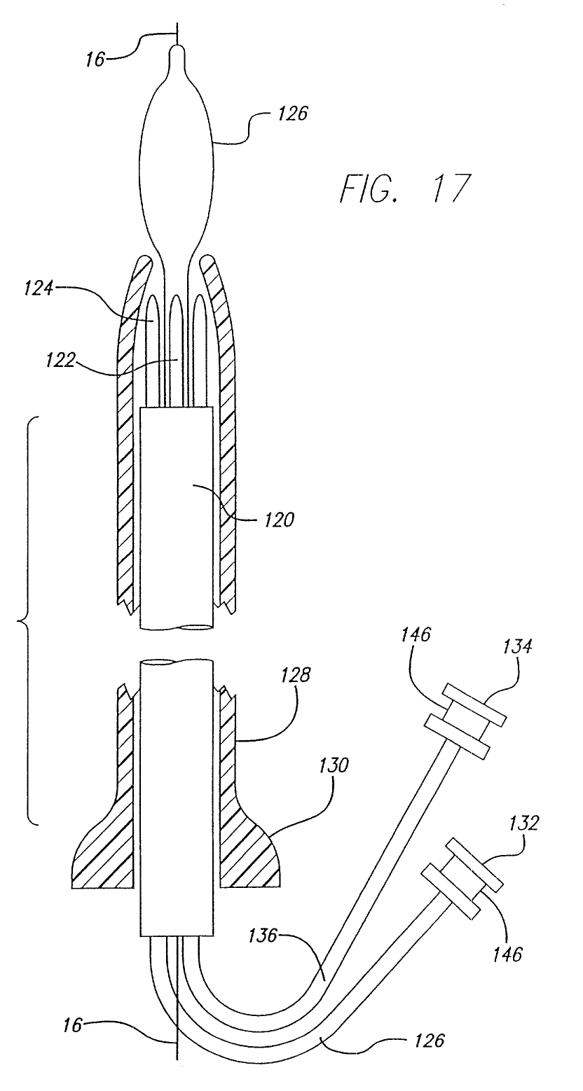

Referring now to FIG. 17, in a fifth embodiment there is shown a system for preventing blood from flowing around the outside of a graft. A delivery catheter 120 has a proximal end and a distal end that may be positioned within a graft. One or more elongate tubular members 122, or hypotubes, each have a proximal end and distal end 124 for placement in the lumen of the delivery catheter. Preferably, a plurality of hypotubes 122 are arranged in a circumferential array about the catheter 120. In one embodiment shown in FIG. 17, three hypotubes are used; however, more or fewer hypotubes maybe used. The hypotubes are preferably formed from stainless steel or a shape memory alloy. The distal end 124 of each hypotube 122 is sharpened for puncturing a wall of the graft. Furthermore, distal ends 124 of hypotubes 122 are each pre-formed into an arcuate configuration. The amount of curvature at distal ends 124, and the length of the portion of hypotube 122 experiencing curvature, may vary depending on clinical needs. However, in this figure hypotubes 122 are retained in a substantially straight configuration as they have yet to be deployed. The proximal end of each hypotube is preferably secured to hose 136 for aiding in axial control of the hypotubes. The point at which the hypotubes are secured to hose 136 may be either inside or outside of catheter 120. The proximal end of hose 136 preferably extends out of the proximal end of catheter 120.

A balloon catheter 126 has a proximal end and a distal end positionable within delivery catheter 120. The distal end of the balloon catheter, which is inflatable, may be extended out of the distal end of delivery catheter 120. Preferably, the system further includes elongate tubular jacket 128 that encapsulates hypotubes 122 and the distal end of delivery catheter 120. At least a portion of hypotubes 122 may be contained within delivery catheter 120. The jacket has retraction handle 130 at its proximal end for moving the jacket relative to delivery catheter 120. The distal end of the delivery catheter is positionable within the jacket, and may be extended beyond the distal end ofjacket 128.

A first luer fitting 146 is attached to the proximal end of balloon catheter 126. A balloon injection port 132 located on first luer fitting 146 is in fluid communication with the interior of balloon catheter 126. The first luer fitting aids in the injection of a substance, such as saline, to cause inflation of balloon catheter 126. A second luer fitting 146 is attached to the proximal end of hose 136. A therapeutic injection port 134 located on second luer fitting 146 is in fluid communication with hypotubes 122 via hose 136. The system is designed so that it may be fed over guide wire 16. The guide wire can pass through a lumen within the balloon catheter.

Preferably, hypotube 122 has a length of the distal arcuate portion ranging from 3 mm (0.12) to 30 mm (1.2).

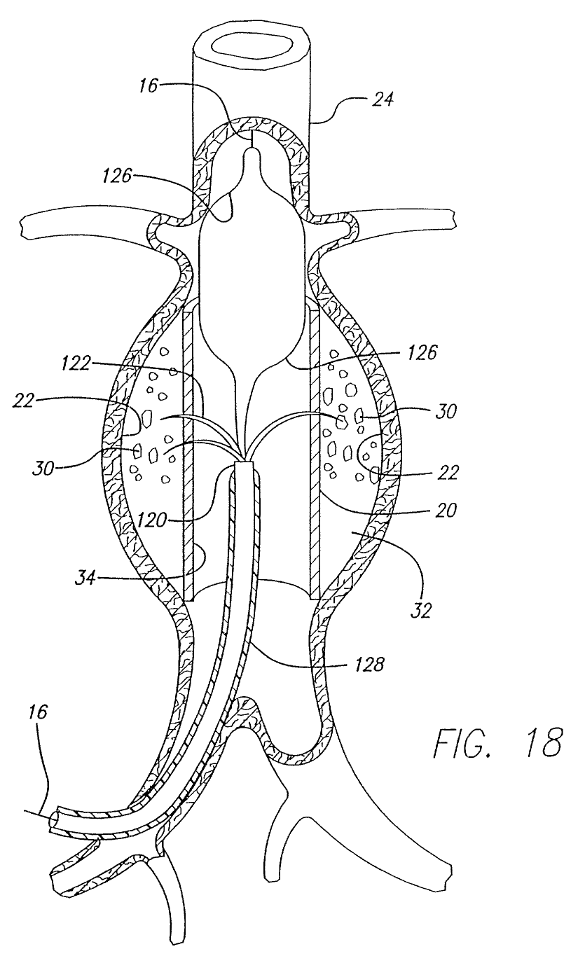

Referring now to FIG. 18, in use, the repair system is advanced through a percutaneous cut-down to a desired site within graft 20 via guide wire 16. The procedure is performed with fluoroscopic visualization. Radiopaque markers identify distal ends 124 of hypotubes 122. In one embodiment, one marker is provided for each hypotube. After the distal portion of elongate tubular jacket 128 is positioned at a desired location within a patient's vasculature, balloon catheter 126 is itself properly positioned. The elongate tubular jacket 128 can be delivered with balloon catheter 126 already inside. Alternatively, the balloon catheter 126 can be delivered after the elongate tubular jacket 128. The balloon catheter 126 can then be inflated at its distal end. The inflation be accomplished by injecting saline or another suitable substance into balloon catheter 126 via balloon injection port 132. The balloon catheter 126 is inflated at its distal end until it comes into apposition with a body vessel inner wall.

After the inflation step, jacket 128 is then retracted, thus exposing the sharpened distal ends of hypotubes 122. The distal ends of the hypotubes assume their preformed arcuate configurations. The hypotubes are then advanced, preferably by advancing hose 136, until their distal ends penetrate the graft and enter space 32 between wall 34 of the graft and wall 22 of vessel 24 (e.g., aneurysm sac 22). A desired substance, such as embolic material 30, is then injected via therapeutic injection port 134 and the hypotubes into the aneurysm sac. The system may then be removed from the treatment area. Thus, the repair of perigraft flow is accomplished in a relatively safe, easy, and efficient manner.

It should be noted that the use of balloon catheter 126 is not necessary in all circumstances; however, when inflated the balloon catheter helps to stabilize delivery catheter 120 from axial migration. The inflated balloon catheter also serves the purpose of centering the delivery catheter within vessel 24 so that hypotubes 122 are targeted and evenly distributed along the circumference of the vessel.

Referring now to FIG. 19, in a sixth embodiment there is shown a system for rapidly delivering coils to a treatment site of a patient. As explained above, many existing coil delivery systems are designed to deliver only one coil at a time. This can slow down the treatment process. Additionally, some systems that use saline to deliver emboli are prone to a relatively high risk of causing hypervolemia from over-infusing the patient with saline. It would therefore be desirable to rapidly deliver coils while minimizing risk to the patient.

The system includes delivery catheter 140 having a proximal end and a distal end positionable within a vessel at a treatment site. The catheter further includes a first lumen 142. A mandrel 144 is provided having a proximal end and a distal end for placement in the first lumen of the catheter. A plurality of hollow coils 146 are included for placement on the mandrel. Preferably, mandrel 144 has a diameter ranging from 0.076 mm (0.003 in) to 0.508 mm (0.020 in) and a length ranging from 20 cm (7.9 in) to 100 cm (39.4 in).

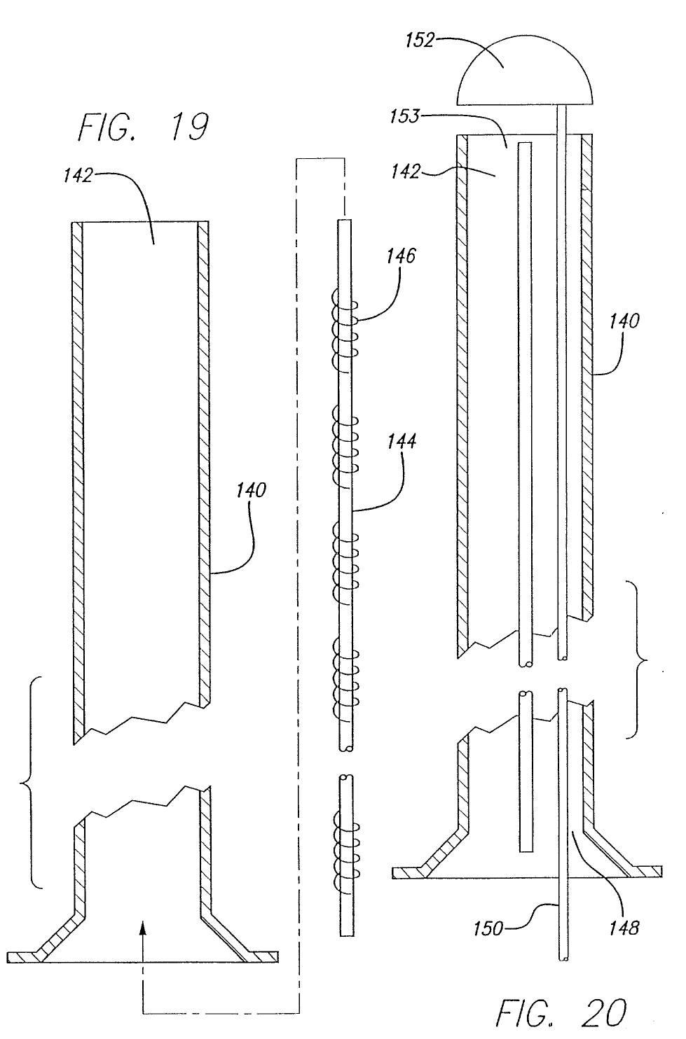

In one preferred embodiment depicted in FIG. 20, delivery catheter 140 further includes second lumen 148. A wire 150 is provided that is positionable within the second lumen and designed to slide therein. The wire 150 is attached to movable cover 152 covering distal exit port 153 in communication with the first lumen. Pushing and pulling the wire 150 aids in opening and closing the cover. The cover helps prevent coils 146 from coming out of the catheter at the wrong time.

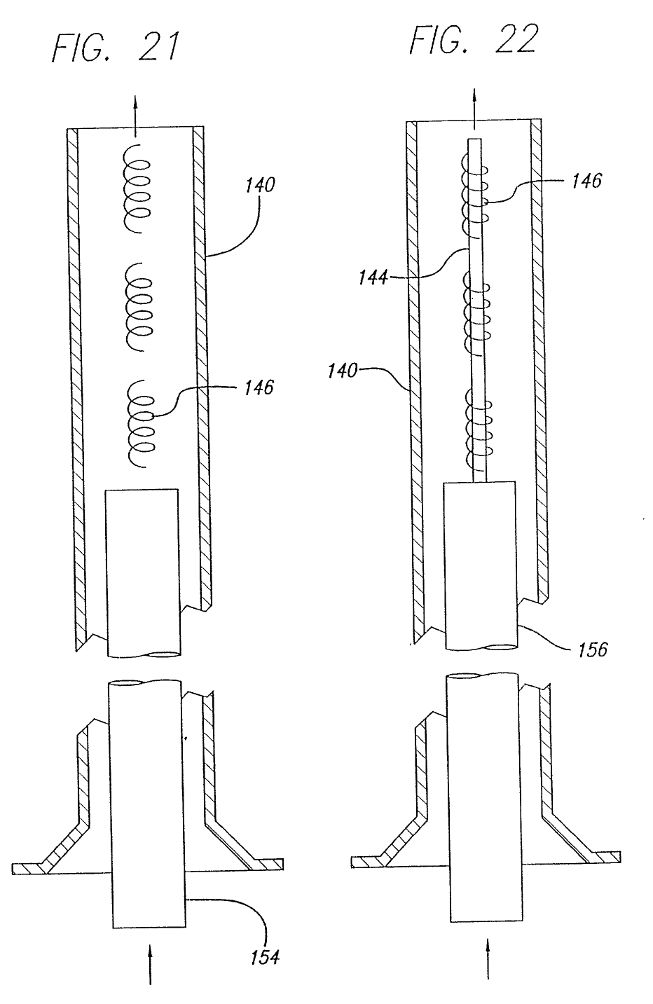

Referring now to FIGS. 19 and 21, in use, the distal end of delivery catheter 140 is advanced to the treatment site. A plurality of hollow coils 146 are strung end-to-end over mandrel 144. The coils may be any of a number of embolic coils known in the art. The coils 146 may be formed from stainless steel, platinum alloy, or any other suitable material. The coils 146 preferably each have a length ranging from 5 mm (0.2 in) to 50 mm (2.0 in), and a diameter ranging from 0.254 mm (0.010 in) to 0.965 mm (0.038 in). The mandrel and coils are inserted into the proximal end of the delivery catheter. The mandrel is then removed from the delivery catheter. The coils 146 remain within the catheter in an end-to-end configuration. The coils 146 can be slightly curved or the catheter may be temporarily blocked at one end in order to help the coils stay in place within the catheter.

Next, guide wire 154 is inserted into the proximal end of the delivery catheter. The guide wire is advanced such that the coils are forced out of the distal end of the delivery catheter. Similarly, this procedure can be used with catheter 140 embodied in FIG. 20.

Referring to FIGS. 19 and 22, in a related method of use, the distal end of delivery catheter 140 is advanced to the treatment site. A plurality of hollow coils 146 are strung over mandrel 144. The mandrel and coils are again inserted into the proximal end of delivery catheter 140. The mandrel is left within the delivery catheter. Next, tube 156 is inserted into the proximal end of delivery catheter 140. The tube 156 is then advanced distally over mandrel 144 and into engagement with the most proximal coil 146. The operator continues to slide tube 156 distally over mandrel 144 such that the most proximal coil is moved in a distal direction. The coils are, in turn, forced out of the distal end of the delivery catheter, beginning with the most distal coil. This embodiment may be used to accomplish the inducement of thrombosis or the repair of perigraft flow in a relatively safe, easy, and efficient manner. Similarly, this procedure can be used with catheter 140 embodied in FIG. 20.

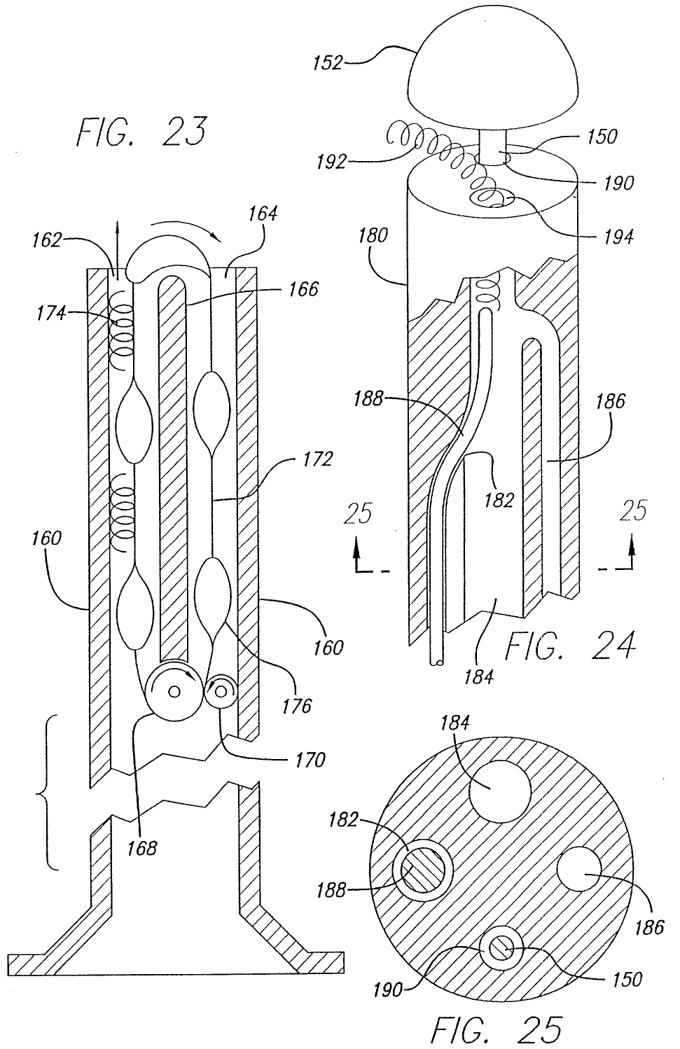

Turning now to FIG. 23, in a seventh embodiment, an apparatus for delivering embolic material to a treatment site of a patient includes delivery catheter 160 having a proximal end and a distal end positionable within a vessel at the treatment site. The catheter further includes first lumen 162 having a distal portion and second lumen 164 having a distal portion. A separating member 166 separates the distal portion of the first lumen from the distal portion of the second lumen. A drive pulley 168 is operatively associated with the delivery catheter. A pinch roller 170 is operatively associated with the delivery catheter and preferably located proximate the drive pulley. Preferably, drive pulley 168 and pinch roller 170 are located near the proximal end of separating member 166. A conveyor line 172 is provided in the shape of a loop. The conveyor line encircles the separating member 166. The conveyor line is in apposition with the drive pulley and the pinch roller and is disposed therebetween. As the drive pulley rotates in a clockwise direction, the conveyor line likewise rotates in a clockwise direction. This causes the pinch roller to rotate in a counterclockwise direction. The conveyor line is thus stabilized between the drive pulley and the pinch roller. The conveyor line is thus capable of frictionally engaging embolic material, such as embolic coils 174, and delivering it to a treatment site of a patient. It is contemplated that additional rotating drive pulleys and pinch rollers could be added to the apparatus to aid in routing the conveyor line. The conveyor line is preferably flexible and can be constructed from nitinol wire, monofilament plastic, multifilament yarn (e.g., Spectra HDPE or Dacron polyester), bead chain, or other suitable materials.

In one preferred embodiment, conveyor line 172, which is preferably flexible, includes a plurality of conveying elements 176 for moving embolic material 174 through the first lumen. The conveying elements may be shaped into discs, bumps 176, or other suitable shapes, and preferably have compliant surfaces so that they can pass between drive pulley 168 and pinch roller 170. The bumps are preferably tapered at their leading edges as shown. The drive pulley and the pinch roller may also have compliant surfaces so that the bumps may easily pass therebetween. The embolic material becomes trapped between bumps 176 and delivery catheter 160. Although only one embolic coil is depicted between a pair of bumps, it is contemplated that more than one coil could be positioned therebetween. Thus, this embodiment may be used to accomplish the inducement of thrombosis or the repair of perigraft flow in a relatively safe, easy, and efficient manner.

Preferably, conveyor line 172 has a cicumferential length that is about two to two and one-half times the length of the catheter. The first lumen 162 can have a diameter ranging from 0.254 mm (0.010 in) to 1.58 mm (0.062 in). The second lumen 164 can have a diameter ranging from 0.254 mm (0.010 in) to 1.58 mm (0.062 in).

Described in general terms, a method contemplated by the present invention includes advancing the distal end of a catheter to a treatment site of a patient. Embolic material is introduced into the proximal end of the catheter. Portions of embolic material are advanced out of the distal end of the catheter in rapid succession. One way in which this can be accomplished is by activating conveyor line 172 to rotate. The conveyor line 172 could be powered by an electric motor, by an external crank, or by any other suitable means. In one embodiment, the embolic material could be introduced into the proximal end of the catheter before the distal end of the catheter is advanced to the treatment site.

Referring now to FIGS. 24 and 25, in an eighth embodiment, a system for delivering embolic material to a treatment site of a patient includes delivery catheter 180 having a proximal end and a distal end positionable within a vessel at the treatment site. The delivery catheter further includes first lumen 182 having a distal portion, second lumen 184 having a distal portion, and third lumen 186 having a distal portion. The first lumen, the second lumen, and the third lumen are substantially parallel to one another for at least a portion of their lengths. Furthermore, the first lumen, the second lumen, and the third lumen are in fluid communication with each other at their distal portions, as shown in FIG. 24. Notably, the first lumen 182 and third lumen 186 preferably terminate at their distal ends before reaching the distal end of delivery catheter 180. However, second lumen 184 extends to the distal end of delivery catheter 180 at which point the second lumen terminates at distal exit port 194. An elongate member, such as a guide wire or pushrod 188, is provided for sliding within the first lumen and at least the distal portion of the second lumen. Preferably, the pushrod is flexible so that it may assume an arcuate configuration as depicted in FIG. 24.

The delivery catheter 180 preferably further includes fourth lumen 190 extending to the distal end of the delivery catheter. Awire 150 is provided that is positionable within the fourth lumen. A movable cover 152 for covering the second lumen is attached to the wire. Pushing and pulling the wire aids in opening and closing the cover. The cover helps prevent embolic material, such as coils 192, from coming out of exit port 194 of the catheter at the wrong time.

In use, embolic material 192 is introduced through second lumen 184. Exit port 194 is closed by cover 152. A liquid, preferably saline, is then pumped into the second lumen. This moves the embolic material toward the distal end of catheter 180. Saline then returns proximally through third lumen 186; however, the third lumen is either too small in diameter for the embolic material to follow suit, or the third lumen is covered by a strainer or similar device.

After embolic material 192 reaches exit port 194, the operator opens cover 152 by advancing wire 150 distally. The embolic material is then forced out of the exit port by advancing pushrod 188 in the distal direction. Alternatively, a small amount of saline may be delivered through second lumen 184 in order to force the embolic material out of the exit port. Alternatively, it is contemplated that the system could be implemented without first lumen 182 and without elongate member 188. One way the system could be implemented in this manner is by using saline to force the embolic material out of exit port 194. Another way the system could be implemented in this manner is by using a pushrod or other elongate member within second lumen 184.

As mentioned above, some systems that use saline to deliver emboli are prone to a relatively high risk of causing hypervolemia from over infusing the patient with saline. Hypervolemia is an abnormal increase in the volume of circulating blood. One of the salient features of this embodiment is that the saline that is routed proximally through third lumen 186 never reaches the patient. Consequently, the volume of saline delivered to the patient is reduced. This embodiment may be used to accomplish the inducement of thrombosis or the repair of perigraft flow in a relatively safe, easy, and efficient manner.

Preferably, first lumen 182 has a diameter ranging from 0.254 mm (0.010 in) to 1.01 mm (0.042 in), second lumen 184 has a diameter ranging from 0.254 mm (0.010 in) to 1.01 mm (0.042 in), and third lumen 186 has a diameter ranging from 0.254 mm (0.010 in) to 1.01 mm (0.042 in).

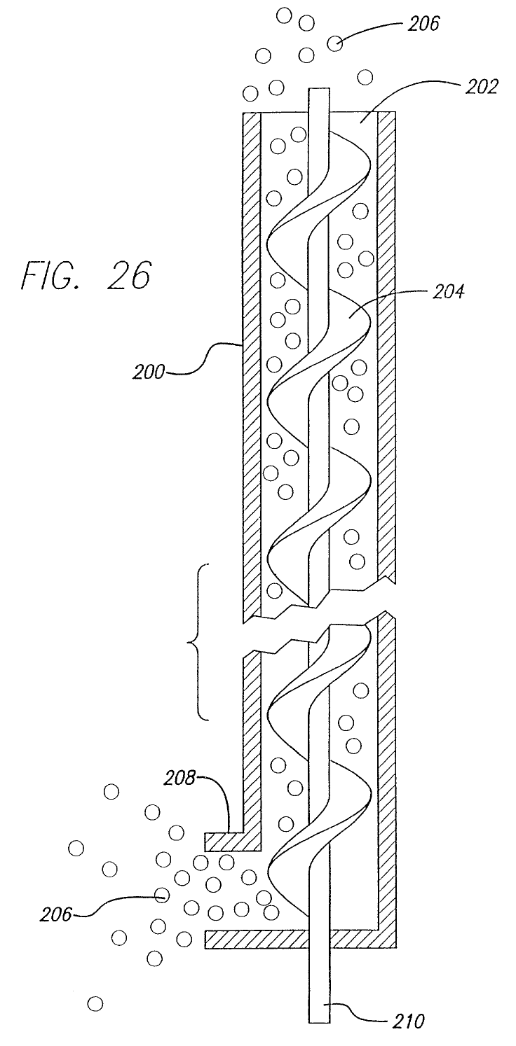

Referring to FIG. 26, in a ninth embodiment, an apparatus for delivering embolic material to a treatment site of a patient includes delivery catheter 200 having a proximal end and a distal end positionable within a vessel at the treatment site. The catheter further includes lumen 202 having a distal portion. A screw 204, or Archimedes screw, is disposed within the lumen. The screw 204 preferably extends for substantially the entire length of lumen 202. The screw is operatively associated with the catheter, and can be rotated within the lumen to deliver embolic material to a treatment site of a patient. The screw can be a flexible plastic extrusion. Alternatively, screw 204 can be made out of another suitable material such as metal. Generally, the pitch of an Archimedes screw is of the same order of its diameter.

Preferably, screw 204 has a diameter ranging from 0.2 mm (0.008 in) to 1 mm (0.04 in) and a length ranging from 30 cm (11.8 in) to 120 cm (47.2 in).

In use, saline and embolic particles 206 are introduced into lumen 202 via loading hopper 208. The screw 204 is rotated such that at least some of the embolic material is forced out of the distal end of the catheter. The screw can be rotated by turning handle 210 by hand. The handle is preferably located at the proximal end of catheter 200. Alternatively, a motor or other appropriate means can be implemented for rotating the screw. A seal is located at the proximal end of the catheter to prevent leakage. When the screw rotates, this forces embolic material out of the distal end of the catheter. Notably, the screw 204 propels the embolic material without over-infusing the patient with saline, thus avoiding the complications associated with hypervolemia. This embodiment may be used to accomplish the inducement of thrombosis or the repair of perigraft flow in a relatively safe, easy, and efficient manner.

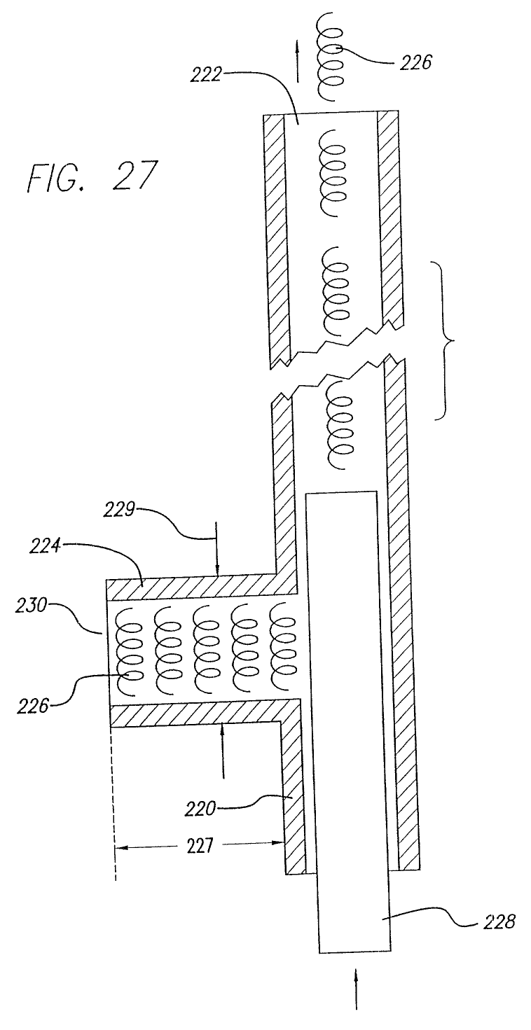

Referring now to FIG. 27, in a tenth embodiment, a system for delivering embolic material to a treatment site of a patient includes delivery catheter 220 having a proximal end and a distal end positionable within a vessel at the treatment site. The catheter further includes lumen 222 having a distal portion. A clip 224 for holding embolic material is in fluid communication with the catheter. The clip may be designed so that it is detachable. As shown in the figure, the clip is capable of holding a plurality of emboli 226, or other embolic material. An elongate member, such as pusher 228, is provided and may be slidably disposed within the lumen. The pusher may be made so that it is flexible.

Preferably, clip 224 has height 227 ranging from 1 cm (0.39 in) to 15 cm (5.9 in), length 229 ranging from 5 mm (0.2 in) to 50 mm (2.0 in), and a width ranging from 0.254 mm (0.010 in) to 1.58 mm (0.062 in). However, the shape and dimensions of clip 224 may vary.

In use, clip 224 is filled with emboli 226. The catheter 220 may be filled with emboli positioned end-to-end inside lumen 222. The distal portion of the catheter is then advanced to a treatment site of a patient. In order to deliver a single embolus from the distal portion of the catheter, pusher 228 is inserted into the proximal portion of the catheter and advanced approximately the length of one embolus. This is repeated until the distal end of the pusher is distal of the clip. The pusher may then be retracted to a position proximal of the clip. At this point an embolus from the clip moves into the lumen. A new embolus may introduced through loading chamber 230. In order to deliver another embolus to the treatment site, the pusher is advanced again. The process may then be repeated as necessary. It is contemplated the system could be operated without the clip. The emboli would then have to be individually loaded into the proximal end of the catheter. This embodiment may be used to accomplish the inducement of thrombosis or the repair of perigraft flow in a relatively safe, easy, and efficient manner.

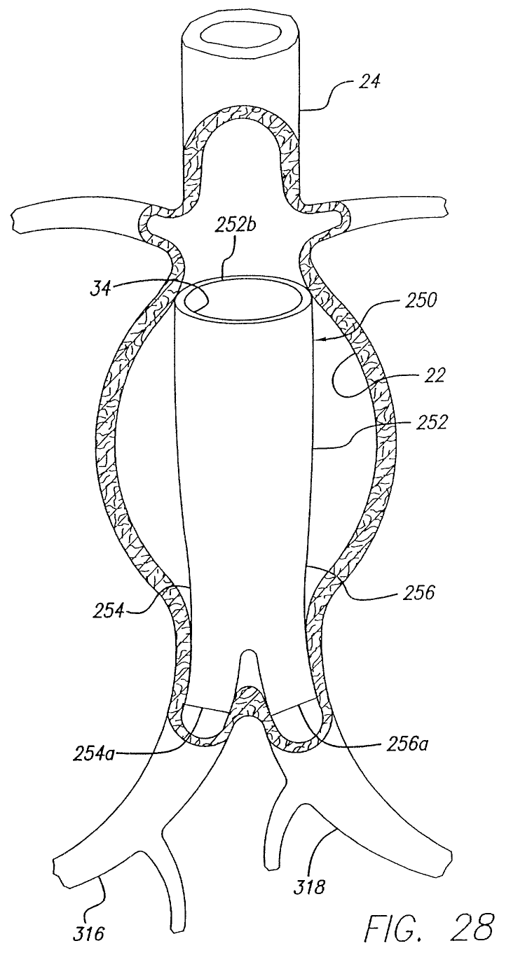

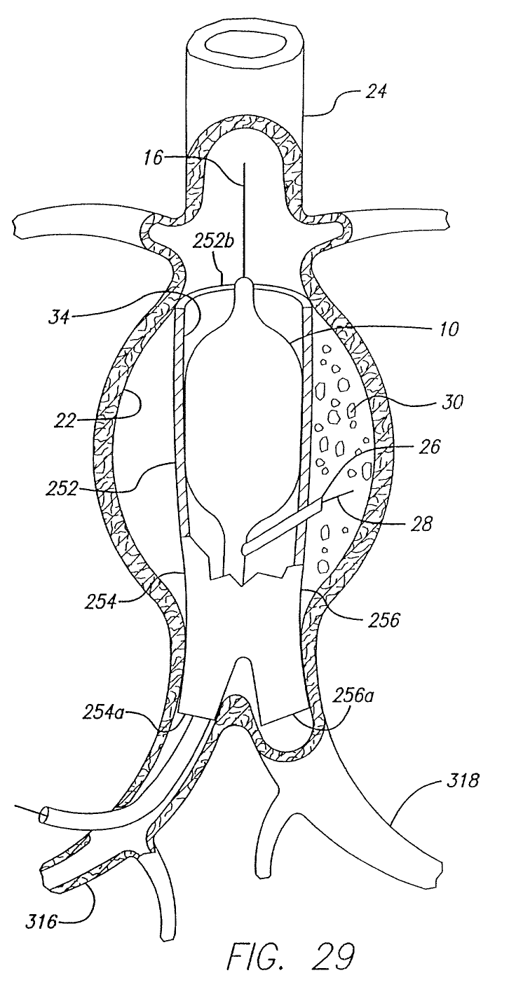

Turning now to FIGS. 28-30, it is contemplated that the embodiments described herein with respect to tubular grafts may be utilized in conjunction with other types of grafts as well. For example, FIG. 28 depicts bifurcated unibody graft 250. The graft includes trunk 252, first leg 254, and second leg 256. The distal end 252b of the trunk is implanted in a non-dilated portion of a vessel such as abdominal aorta 24. The proximal end 254a of the first leg is implanted in an undilated portion of ipsilateral iliac artery 316. The proximal end 256a of the second leg is implanted in an undilated portion of contralateral iliac artery 318. The unibody graft can be used in conjunction with the embodiments of the present invention that utilize grafts. One such example of perigraft flow repair in conjunction with a bifurcated graft is depicted in FIG. 29.

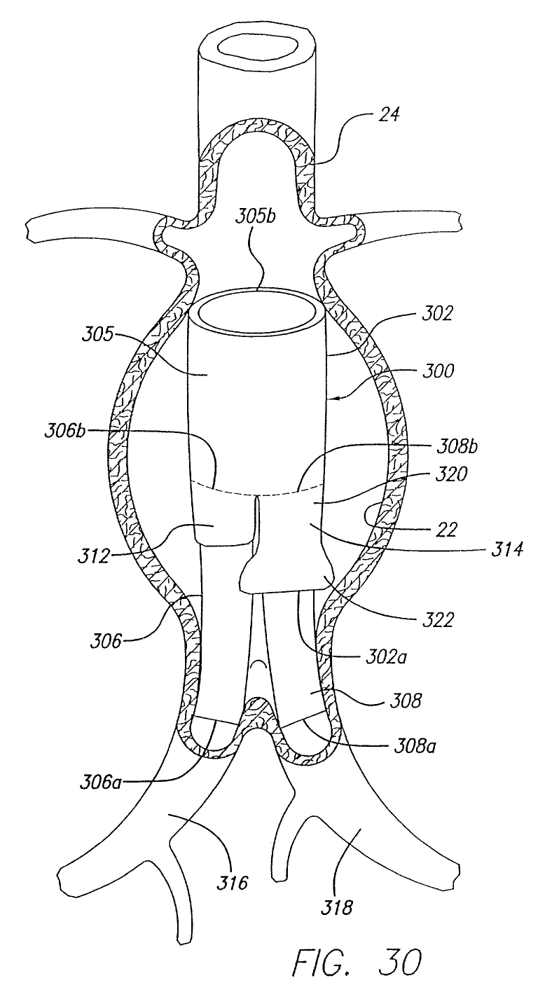

Referring to FIG. 30, bifurcated modular graft 300 is illustrated as implanted to repair an aneurysm such as abdominal aorta aneurysm 22. The graft 300 includes first graft component 302 having proximal end 302a and distal end 302b. The graft 300 also includes second graft component 306, often referred to as the ipsilateral extension, and third graft component 308, often referred to as the contralateral extension. The distal end 305b of trunk 305 is implanted in a non-dilated portion of a vessel such as abdominal aorta 24. The distal end 306b of the second graft component, or ipsilateral extension, is connected to the first graft component at ipsilateral docking site 312. The distal end 308b of the third graft component, or contralateral extension, is connected to the first graft component at contralateral docking site 314. The proximal end 306a of the second graft component is implanted in an undilated portion of ipsilateral iliac artery 316. The proximal end 308a of the third graft component is implanted in an undilated portion of the contralateral iliac artery 318. The contralateral leg 320 of the first graft component terminates in bell-bottom 322. The bell-bottom aids in the surgical implantation and manipulation of the modular graft. The modular graft can also be used in conjunction with the embodiments of the present invention that utilize grafts.

While the invention has been illustrated and described herein in terms of its use as a method and apparatus for the repair of perigraft flow, it will be apparent to those skilled in the art that the invention can be used in other instances. For example, the tubular grafts may be of the bifurcated type. Additionally, the system may be used to deliver fasteners, gene therapy drugs, plaque dissolving agents, tissue adhesives for laminating dissection or other substances. Other modifications and improvements may be made without departing from the scope of the invention.

What is claimed is:

1. A system for preventing blood from flowing around the outside of a graft, comprising:
a catheter having a proximal end and a distal end;
an elongate tubular member having a proximal end and a distal end for placement in a lumen of the catheter; and
a needle for puncturing a wall of the graft, the needle positionable within the elongate tubular member and deflectable such that the needle is angled into a position for piercing the wall of the graft.

2. The system of claim 1, wherein the catheter may be inflated at its distal end.

3. The system of claim 1, wherein the catheter includes a support structure for housing the elongate tubular member.

4. The system of claim 1, wherein the distal end of the elongate tubular member is deflectable.

5. The system of claim 1, wherein the needle is hollow for injecting embolic material through the graft wall and into a space between the graft and an aneurysm sac.

6. The system of claim 1, wherein the needle is pre-formed into a naturally arcuate configuration.

7. A method for preventing blood from flowing around the outside of a graft, utilizing a system including a catheter, an elongate tubular member, and a needle, comprising the steps of:
accessing a leaking site of a vessel with a distal end of the catheter by feeding the catheter along a guide wire that is positioned within the graft;
inflating the catheter;
extending the needle such that the needle pierces a wall of the graft;
injecting embolic material into a space between the outside of the graft and the vessel via the needle; and
removing the catheter.

8. A system for preventing blood from flowing around the outside of a graft, comprising:
a main catheter having a proximal end and a distal end; and
an injection catheter, the injection catheter having a proximal end and a distal end positionable within the main catheter, whereby the injection catheter may be advanced relative to the main catheter for piercing a wall of the graft.

9. The system of claim 8, wherein the distal end of the main catheter is curved.

10. The system of claim 8, wherein the distal end of the main catheter is deflectable.

11. The system of claim 8, wherein the injection catheter includes a needle-like injection tip for facilitating the injection of embolic material.

12. The system of claim 11, wherein the distal end of the injection catheter is deflectable.

13. The system of claim 8, further including a guide wire, wherein the distal end of the injection catheter is curved.

14. A method for preventing blood from flowing around the outside of a graft, utilizing a system including a main catheter, an injection catheter, and a guide wire, comprising the steps of:
advancing the system to a location adjacent the graft;
retracting the guide wire to allow the main catheter to recover to a set geometry;
advancing the injection catheter, the injection catheter including a needle-like injection tip for facilitating the injection of embolic material relative to the main catheter so that a distal end of the injection catheter penetrates a wall of the graft;
injecting embolic material into a space between the outside of the graft and a vessel;
retracting the injection catheter; and
removing the system.

15. A system for preventing blood from flowing around the outside of a graft, comprising:
a guide catheter, the guide catheter having a proximal end and a distal end;
a delivery catheter, the delivery catheter having a proximal end and a distal end and positionable within the guide catheter; and
a bendable member for stabilizing the guide catheter, so that the delivery catheter may be advanced relative to the guide catheter and toward a wall of the graft.

16. The system of claim 15, wherein the bendable member operates to deflect the guide catheter when inflated.

17. The system of claim 15, further including a guide wire, the guide wire having a proximal end and a distal end and positionable within the delivery catheter.

18. The system of claim 17, wherein the distal end of the guide wire includes a sharp tip for piercing the wall of the graft.

19. The system of claim 17, wherein the guide wire is pre-formed into an arcuate configuration.

20. The system of claim 15, wherein the distal end of the guide catheter is deflectable.

21. The system of claim 20, wherein the guide catheter is pre-formed into an arcuate configuration.

22. The system of claim 15, wherein the distal end of the delivery catheter includes a needle-like injection tip for facilitating the injection of embolic material.

23. The system of claim 22, wherein the distal end of the delivery catheter is deflectable.

24. The system of claim 15, wherein the bendable member includes an eccentric balloon.

25. The system of claim 15, wherein the bendable member includes an inner tension device.

26. A method for preventing blood from flowing around the outside of a graft, utilizing a system including a guide catheter, a delivery catheter, and a bendable member for deflecting the guide catheter, comprising the steps of:
advancing the guidewire to a location adjacent the graft;
advancing the guide catheter to the location adjacent the graft;
advancing the delivery catheter through the guide catheter until the delivery catheter touches a wall of the graft;
advancing the guide wire, wherein a distal end of the guide wire includes a sharp tip for piercing the wall of the graft, relative to the delivery catheter so that the guide wire pierces the wall of the graft;
advancing the delivery catheter over the guide wire and through the wall of the graft while stabilizing the guide catheter with the bendable member;
removing the guide wire;
stabilizing the delivery catheter with the bendable member;
injecting embolic material via the delivery catheter into a space between the outside of the graft and a vessel; and
removing the system.

27. The method of claim 26, wherein the bendable member is connected to the guide catheter, and the step of stabilizing the delivery catheter is accomplished by stabilizing the guide catheter, and the step of removing the guide catheter occurs after the step of stabilizing the delivery catheter.

28. The method of claim 27, wherein the bendable member is a deflection element, further including the step of activating the deflection element so that the delivery catheter is deflected.

29. A system for preventing blood from flowing to a portion of a vessel, comprising:
a graft including a superior end and an inferior end;
a catheter, having a proximal end and a distal end positionable within the graft, wherein the catheter may be inflated at its distal end; and
a delivery catheter, wherein the delivery catheter is positionable between the graft and the vessel and facilitates the injection of embolic material.

30. The system of claim 29, wherein the catheter fits over a guide wire.

31. A method for preventing blood from flowing to a portion of a vessel, utilizing a system including a graft, the graft having a superior end and at least one inferior end, a catheter, having a proximal end and a distal end positionable adj acent the graft, and a delivery catheter, having a proximal end and a distal end, comprising the steps of:
accessing a desired site with a system via a first guide wire;
inflating the catheter;
implanting the superior end of the graft;
inserting a second guide wire between the system and wall of a vessel;
deploying the inferior end of the graft;
advancing the delivery catheter over the second guide wire until the distal end of the delivery catheter is between a wall of the graft and the wall of the vessel;
removing the second guide wire from the delivery catheter;
injecting embolic material via the delivery catheter into a space between the wall of the graft and the vessel;
removing the delivery catheter;
deflating the catheter;
repositioning the catheter inside the inferior end of the graft;
inflating the catheter to implant the inferior end of the graft;
deflating the catheter; and
removing the catheter along with the first guide wire.

32. A system for preventing blood from flowing around the outside of a graft, comprising:
first means for piercing a graft, positionable within the graft;
second means for deflecting first means such that first means is in a position to pierce the graft; and
third means for advancing first means such that first means pierces the graft, thus creating an aperture in the graft.

33. The system of claim 32, further including fourth means for injecting embolic material through the aperture.

34. A method for preventing blood from flowing around the outside of a graft, utilizing first means for piercing the graft, second means for deflecting first means, third means for advancing first means, and fourth means for inj ecting embolic material through the aperture; comprising the steps of:
positioning adjacent the graft first means for piercing the graft;
positioning adjacent the graft second means for deflecting first means;
activating second means such that first means is in a position to pierce the graft;
advancing first means such that first means pierces the graft, thus creating an aperture in the graft; and
injecting embolic material through the aperture via fourth means.

35. A system for preventing blood from flowing around the outside of a graft, comprising:
a delivery catheter having a proximal end and a distal end positionable adjacent the graft; and
an elongate tubular member having a proximal end and a distal end for placement in a lumen of the catheter, the distal end of the elongate tubular member being sharpened for puncturing a wall of the graft, the distal end of the elongate tubular member further being pre-formed into an arcuate configuration.

36. The system of claim 35, further including a balloon catheter having a proximal end and a distal end positionable within the delivery catheter, the distal end being inflatable.

37. The system of claim 35, further including an elongate tubular jacket, the distal end of the delivery catheter being positionable within the elongate tubular jacket.

38. The system of claim 35, further including a circumferential array of hypotubes.

39. The system of claim 38, further including a cord, wherein a hypotube is secured to the cord to aid in axial control of the hypotube.

40. The system of claim 37, wherein the elongate tubular jacket includes a proximal end and a distal end, the jacket further including a retraction handle at its proximal end for moving the jacket.

41. A method for preventing blood from flowing around the outside of a graft, utilizing a system including a delivery catheter, an elongate tubular member having a sharpened distal tip, and an elongate tubular jacket, comprising the steps of:
advancing the system to a location adjacent the graft;
retracting the jacket and exposing the sharpened distal tip, wherein the elongate tubular member assumes an arcuate configuration at its distal end;
advancing the sharpened distal tip of the elongate tubular member until the tip pierces the wall of the graft;
injecting a desired substance via the elongate tubular member into a space between the outside of the graft and a vessel; and
removing the system.

42. The method of claim 41, wherein the system further includes a balloon catheter, further including the step of inflating the balloon catheter.

43. A system for delivering coils to a treatment site of a patient, comprising:
a delivery catheter having a proximal end and a distal end positionable within a vessel at a treatment site, the catheter further including a first lumen;
a mandrel having a proximal end and a distal end for placement in the first lumen of the catheter; and
a plurality of hollow coils for placement on the mandrel.

44. The system of claim 43, wherein the delivery catheter further includes a second lumen, further comprising:
a wire positionable within the second lumen; and
a cover for covering the first lumen, the cover attached to the wire.

45. A method for delivering coils to a treatment site of a patient, utilizing a system including a delivery catheter, a mandrel, and a plurality of hollow coils, comprising the steps of:
advancing the distal end of the delivery catheter to the treatment site;
stringing a plurality of hollow coils over the mandrel;
inserting the mandrel and coils into the proximal end of the delivery catheter;
removing the mandrel from the delivery catheter;
inserting a guide wire into the proximal end of the delivery catheter; and
advancing the guide wire such that the coils are forced out of the distal end of the delivery catheter.

46. A method for delivering coils to a treatment site of a patient, utilizing a system including a delivery catheter, a mandrel, and a plurality of hollow coils, comprising the steps of:
advancing the distal end of the delivery catheter to the treatment site;
stringing a plurality of hollow coils over the mandrel;
inserting the mandrel and coils into the proximal end of the delivery catheter;
inserting a tube into the proximal end of the delivery catheter; and
advancing the tube over the mandrel such that the coils are forced out of the distal end of the delivery catheter.

47. An apparatus for delivering embolic material to a treatment site of a patient, comprising:
a delivery catheter having a proximal end and a distal end positionable within a vessel at the treatment site, the catheter further including a first lumen having a distal portion and a second lumen having a distal portion;
a separating member separating the distal portion of the first lumen from the distal portion of the second lumen;
a drive pulley operatively associated with the delivery catheter;
a pinch roller operatively associated with the delivery catheter and proximate the drive pulley; and
a conveyor line in the shape of a loop and encircling the separating member, the conveyor line in apposition with the drive pulley and the pinch roller and disposed therebetween.

48. The apparatus of claim 47, wherein the conveyor line includes a plurality of conveying elements for moving embolic material through the first lumen.

49. The apparatus of claim 47, wherein the conveyor line is flexible.

50. The apparatus of claim 48, wherein the conveying elements are shaped like bumps.

51. The apparatus of claim 50, wherein the bumps have compliant surfaces.

52. The apparatus of claim 50, wherein the bumps are tapered.

53. The apparatus of claim 48, wherein the conveying elements are shaped like discs.

54. The apparatus of claim 47, wherein the drive pulley and the pinch roller have compliant surfaces.

55. A method for delivering embolic material to a treatment site of a patient, utilizing a catheter having a proximal end and a distal end, comprising the steps of:
advancing the distal end of the catheter to the treatment site;
introducing embolic material into the proximal end of the catheter; and
advancing portions of embolic material out of the distal end of the catheter in rapid succession.

56. An system for delivering embolic material to a treatment site of a patient, comprising:
a delivery catheter having a proximal end and a distal end positionable within a vessel at the treatment site, the catheter further including a second lumen having a distal portion and a third lumen having a distal portion;
wherein the second lumen and the third lumen are in fluid communication at their distal portions.

57. The system of claim 56, further including:
a first lumen having a distal portion, the distal portion of the first lumen being in fluid communication with the distal portion of the second lumen and the distal portion of the third lumen; and
an elongate member for sliding within the first lumen and at least the distal portion of the second lumen.

58. The system of claim 56, wherein the delivery catheter further includes a fourth lumen, further comprising:
a wire positionable within the fourth lumen; and
a cover for covering the second lumen, the cover attached to the wire.

59. The system of claim 57, wherein the elongate member is flexible.

60. A method for delivering embolic material to a treatment site of a patient, utilizing a catheter having a proximal end and a distal end, comprising the steps of:
advancing the distal end of the catheter to the treatment site;
introducing embolic material into the proximal end of the catheter;
moving the embolic material distally through the catheter by pumping a liquid into the catheter, wherein at least some of the liquid moves proximally through a lumen and never reaches the patient; and
advancing embolic material out of the distal end of the catheter.

61. An apparatus for delivering embolic material to a treatment site of a patient, comprising:
a catheter having a proximal end and a distal end positionable within a vessel at the treatment site, the catheter further including a lumen having a distal portion; and
a screw disposed within the lumen, the screw operatively associated with the catheter.

62. A method for delivering embolic material to a treatment site of a patient, utilizing a catheter having a proximal end and a distal end positionable within a vessel at the treatment site, the catheter further including a lumen having a distal portion, a screw disposed within the lumen, the screw operatively associated with the catheter, comprising the steps of:
introducing embolic material into the lumen of the catheter; and
rotating the screw such that at least some of the embolic material is forced out of the distal end of the catheter.

63. An system for delivering embolic material to a treatment site of a patient, comprising:
a catheter having a proximal end and a distal end positionable within a vessel at the treatment site, the catheter further including a lumen having a distal portion;
a clip for holding embolic material, the clip in fluid communication with the catheter; and
an elongate member that may be slidably disposed within the lumen.
